(19) United States
(12) Patent Application Publication
(10) Pub. No.: US 2022/0033470 A1
(43) Pub. Date: Feb. 3, 2022

(54) Compositions Comprising Sortilin-1

(71) Applicant: Insusense ApS, Copenhagen (DK)

(72) Inventors: Mads Fuglsang KJØLBY, Copenhagen (DK); Anders NYKJÆR, Risskov (DK); Søren THIRUP, Aarhus C (DK)

(21) Appl. No.: 17/413,184

(22) PCT Filed: Dec. 13, 2019
(86) PCT No.: PCT/EP2019/085100

§ 371 Date: Jun. 11, 2021

(30) Foreign Application Priority Data
Dec. 14, 2018 (EP) 18212797.7

(51) Int. Cl. C07K 14/705; A61P 3/10

(52) U.S. Cl. CPC C07K 14/70571; A61K 38/00; A61P 3/10

(57) ABSTRACT

The invention provides composition comprising a fragment, derivative or variant of sortilin, or a sortilin mimetic, for use in medicine, wherein the sortilin mimetic is not SorCS1, SorCS2, SorCSS or SorLA. The composition preferably for use in treating or preventing diabetes mellitus, insulin resistance, obesity, metabolic disorders, polycystic ovary syndrome, non-alcoholic fatty liver disease, retinopathy, neuropathy, nephropathy, Alzheimer's disease and/or cardiovascular disease in a subject in need thereof. Also provided are isolated polypeptides, nucleic acids encoding the polypeptides, vectors, host cells and expression systems containing the nucleic acids and methods for making the polypeptides.

### FIELD OF INVENTION

The present invention relates to pharmaceutical compositions for use in the treatment of diseases including obesity, insulin resistance and diabetes mellitus.

### BACKGROUND

Obesity is a medical condition in which excess body fat has accumulated to the extent that it may have a negative impact on health. With 61.4% of adults in England, for example, being overweight or obese, obesity represents a major public health issue. Obesity is known to increase the risk of a range of health problems, including insulin resistance and the subsequent development of type 2 diabetes mellitus. Insulin resistance occurs when an individual's cells fail to respond normally to the hormone insulin. To overcome this, the pancreas (the organ responsible for producing insulin) compensates by producing more insulin to meet the metabolic demand. Ultimately, the levels of insulin produced are no longer adequate and therefore cellular glucose uptake is impaired, leading to hyperglycaemia and the likely development of diabetes mellitus.

The above diseases are not only an economic burden in their own right but can lead to multiple secondary disease states, further exacerbating the problem. Additionally, current pharmaceutical interventions fail to display adequate efficacy in a large proportion of patients, highlighting the need for novel treatment strategies.

The vacuolar protein sorting 10p domain (Vps10pD) receptor family is a group of five type I membrane homologs, known to include Sort1 (sortilin), SorL1, SorCS1, SorCS2 and SorCS3. This domain family was characterised following the identification of the yeast-sorting protein, Vps10p, which transports enzymes from the trans-Golgi network to the vacuole. These proteins are characterised by a domain within their N terminus that represents a site for ligand binding. Functions known to be attributed to this family of receptors include protein trafficking, lipid metabolism and both intracellular and intercellular signalling in a range of cell types.

In addition to the Vps10pD, SorCS1, SorCS2, SorCS3 all contain a leucine-rich segment between the Vps10pD and the transmembrane domain. SorLA is further characterised by the presence of fibronectin-type III repeats, low-density lipoprotein receptor class A and B repeats and an EGF precursor-type repeat. The peptide sequence homology between SorCS1, SorCS2, SorCS3 and SorLA is high, which is in contrast to a very low homology of these receptors with sortilin (for example the homology between human sortilin and human SorCS1 in the Vpsp10 domain is 25%).

WO 2010/142296 claims a SorCS1-like agent for treating insulin resistance and/or a disease associated with insulin resistance in an individual, wherein said agent is capable of binding to and sensitising the insulin receptor at a SorCS1 binding site. Additionally, WO 2013/156031 claims the use of SorCS1 and SorCS1-like agents in a method of reducing appetite, suppressing hunger, increasing metabolism and treating obesity.

Different splice variants of SorCS1 display distinct patterns of tissue distribution and have been found to be present in tissues involved in glucose metabolism processes, such as adipose tissue, skeletal muscle and p-cells of the pancreas. SorCS1 knock-out mice show a reduced response to insulin when compared to wild-type control mice. Additionally, in a mouse model of diabetes mellitus, the over-expression of soluble SorCS1 reduced plasma levels of both glucose and insulin.

Sortilin is known to be enriched in the nervous system, where it can regulate neuronal development, functionality and integrity, via the binding of neurotrophic factors, for example. It is also known that it plays an important role in controlling metabolic activities. Examples of the latter include the regulation of cholesterol metabolism and regulation of the trafficking of GLUT-4 containing vesicles (Kjolby et al. Curr Atherosclerosis Reports 17(4):496, 2015).

Previous work has suggested a role for sortilin in the cellular signalling pathways associated with diseases such as diabetes and obesity. Sortilin levels have been shown to be modulated by the level of inflammation associated with these diseases. The pro-inflammatory cytokine, TNFα, reduces both mRNA levels and protein levels of sortilin in cultured mouse and human adipocytes, as well as in vivo when injected into mice (Kaddai et al. Diabetologia 52: 932-40, 2009). Sortilin can also influence cytokine secretion: targeting sortilin in immune cells has been proposed to attenuate inflammation (Mortenson et al. J Clin Invest 124(12):5317-22, 2014). Additionally, US 2016/0331746 describes various scaffolds of small molecules capable of binding to the active site of sortilin. Sortilin is involved in the regulation of glucose uptake (Shi & Kandror. Developmental Cell 9:99-108, 2005) and the development of lipid disorder diseases (Gao et al. DNA and Cell Biology 36(12):1050-61, 2017).

Further, plasma sortilin levels have been reported to be a potential biomarker for identifying patients with either coronary heart disease or diabetes mellitus (Oh et al. Cardiovascular Diabetology 16:92, 2017). Patients that showed increased sortilin levels within their plasma, and therefore identifiable as suffering from the above conditions, also displayed enhanced glucose levels suggesting sortilin as a therapeutic target for treating these conditions.

The listing or discussion of an apparently prior-published document in this specification should not necessarily be taken as an acknowledgment that the document is part of the state of the art or is common general knowledge.

### SUMMARY OF INVENTION

The present invention is based on the surprising discovery by the inventors that fragments of sortilin, when administered to diabetic mice, are able to significantly lower blood glucose levels by increasing insulin sensitivity following glucose challenge. This is particularly surprising given the very low homology of sortilin to SorCS1 and related proteins, and the suggestion in the art that downregulation of sortilin may be desirable for treating conditions relating to insulin resistance.

Accordingly, the present invention provides a composition comprising a fragment, derivative or variant of sortilin, or a sortilin mimetic, for use in medicine, wherein the sortilin mimetic is not SorCS1, SorCS2, SorCS3 or SorLA. In particular, the composition may be for use in treating or preventing diabetes mellitus, insulin resistance, obesity, metabolic disorders, polycystic ovary syndrome, non-alcoholic fatty liver disease, retinopathy, neuropathy, nephropathy, Alzheimer's disease and/or cardiovascular disease in a subject in need thereof.

The invention further provides an isolated polypeptide comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23, or an amino acid sequence having greater than 70% homology to said amino acid sequence, or a fragment thereof.

Further provided are nucleic acids that encode the polypeptides of the invention and pharmaceutical compositions comprising the isolated polypeptides or nucleic acids of the invention and medical uses thereof.

The present invention also provides a method of manufacturing polypeptides or fragments thereof according to the invention comprising the step of expressing the nucleic acid of the invention in a host cell or other suitable expression system as indicated herein.

The invention will now be described in more detail with reference to the following figures and examples.

### FIGURES

FIG. 1: Alignment of hSORT1 and mSort1, hSORT1 and hSORCS1.

FIG. 2: Sortilin and insulin receptor binding. Surface plasmon resonance of sortilin (50-500 nM) binding to immobilized (A) human insulin receptor (0.0609 pmol/mm²), and (B) murine insulin receptor (0.0607 pmol/mm²). Sortilin and insulin (C) is able to bind to the insulin receptor at the same time. (D) Co-immunoprecipitation with the insulin receptor and sortilin using DSP crosslinker. Immunoprecipitation of the insulin receptor resulted in co-precipitation of sortilin, and vice versa.

FIG. 3. Effects of exogenous hSortilin given to Lepr^{-/-} (db/db) mice at t=−12 hrs and t=−1 hrs (100 μg/mouse x2). Mice were challenged with an IP dose of glucose (1 mg/g BW) at t=0 min. (A) Plasma glucose levels in the intraperitoneal glucose tolerance test (IPGTT), (B) individual plasma glucose levels in IPGTT, (C) area under the curve (AUC) without baseline, (D) total AUC in IPGTT, and (E) body weight prior to conducting the experiment. Statistics: Student's t-test, and 2-way-ANOVA in panel A. Values are Mean±SEM.

FIG. 4. Effects of treatment with sortilin+/−heparin in diabetic db/db mice on IPGTT response. (A) Initial bodyweight (g) (P=n.s), (B) basal glucose levels (mM) prior to IPGTT, (C) average IPGTT curves, and individual (D) curves. AUC (total) was lower in treated animals (E). Basal insulin levels (P=0.05 (soluble sortilin)) (F), and insulin IPGTT response curves (G-H), and insulin AUC (I). Insulin sensitivity index was calculated, HOMA1-IR (J), QUICKI Index (K), and plasma glucose vs insulin (L).

### DETAILED DESCRIPTION

The following description is presented to enable any person skilled in the art to make and use the invention. Various modifications to the disclosed embodiments will be readily apparent to those skilled in the art.

In a first aspect the present invention provides a composition comprising a fragment, derivative or variant of sortilin, or a sortilin mimetic, for use in medicine, wherein the sortilin mimetic is not SorCS1, SorCS2, SorCS3 or SorLA.

Sortilin is a member of the Vps10p domain (Vps10pD) receptor family and has an amino acid sequence according to SEQ ID NO: 12 or SEQ ID NO: 13. Full length sortilin comprises a signal peptide, a propeptide, the Vps10p domain, a 1000 domain (10CCa+10CCb), a transmembrane domain and a large cytoplasmic tail. The lumenal domain of sortilin has 6 potential N-linked glycosylation sites, whilst the cytoplasmic tail enables for the recruitment of various adapter proteins. Sortilin binds a vast number of ligands and membrane receptors and as a result engages in functions known to be important in cellular signalling and sorting.

The composition of the invention may include, in addition to the active pharmaceutical ingredient(s) (sortilin fragment, derivative, variant or mimetic), pharmaceutically acceptable carriers or excipients as would be understood by a person of skill in the art.

The present compositions may comprise sortilin fragments, derivatives or variants. By ‘fragment’ we intend any sequence of amino acids which partially constitute the full length amino acid sequence of sortilin. Therefore this includes any continuous length of amino acids with 100% identity to a sortilin amino acid sequence. By ‘derivative’ we intend any sequence of natural or non-natural amino acids derived from reasonable modification of either the full length amino acid sequence of sortilin or the aforementioned sortilin fragments. Thus, the derivative may contain modified amino acids, D-amino acids, or amino acid mimics, which may be optimised for improved pharmacological properties, as would be understood by a person of skill in the art. By ‘variant’ we intend any form of sortilin or amino acid with high homology to sortilin and functional equivalent, which includes splice variants and homologues from organisms other than humans, for example, simian, murine or porcine homologues.

The present composition may comprise sortilin mimetics. The term ‘sortilin mimetic’ is intended to refer to any substance which exerts the same function as sortilin, sortilin fragments, derivatives or variants thereof. For example, such compounds may exhibit the same receptor binding capabilities as sortilin, even though they may not share structural homology to sortilin. However, “sortilin mimetic” does not include SorCS1, SorCS2, SorCS3 or SorLA. It is envisaged that these substances may include antibodies, nanobodies and aptamers which are able to interact with the sortilin binding epitope(s) within the insulin receptor. Methods for detecting such an interaction may involve techniques such as phage display, an in vitro screening technique designed for identifying ligands for proteins. The antibody used in the present invention may be, for example, a monoclonal antibody, a humanised antibody, a chimeric antibody, a recombinant antibody or a bispecifc antibody. By ‘bispecific antibody’ we intend an antibody that can simultaneously bind to two different antigens. The latter may be a suitable method to bridge the insulin receptor with endogenous sortilin. Fragments of said antibodies may also be used, specifically, antigen-binding fragments or single chain variable fragments. Preferred fragments will retain some or all the ability of an antibody to selectively bind to its target. By ‘nanobodies’ we intend the resulting protein based on single-domain antibody fragments that contain the unique structural and functional properties of naturally-occurring heavy chain only antibodies. By ‘aptamer’ we intend an oligonucleotide or peptide molecule capable of binding to a specific target molecule. Aptamers can bind to their target with high selectivity and specificity. Target recognition and binding may involve shape-dependent interactions as well as hydrophobic interactions, base-stacking and intercalation. Common targets for these molecules may include proteins, peptides, carbohydrates and small molecules. The methods by which the above antibodies, antibody fragments and aptamers are produced will be well known to the person skilled in the art.

It is envisaged that the present invention may useful for treating a range of diseases. The invention may be for use as a therapeutic and/or a prophylactic treatment. Specifically, the present invention is intended for use in diseases associated with insulin resistance. These diseases may be as a direct result of elevated blood glucose levels or as a secondary cause. Examples of such diseases include, but are not limited to, diabetes mellitus, obesity, metabolic disorders, polycystic ovary syndrome, non-alcoholic fatty liver disease, retinopathy, neuropathy, nephropathy, Alzheimer's disease, cardiovascular disease, stroke, coeliac disease, thyroid disease, mastopathy, muscular conditions, cirrhosis, hyperglycemia, hyperinsulinemia, arteriosclerosis, hypercholesterolemia, hypertriglyceridemia, dyslipidemia, hypercoagulability, hypertension, microalbuminura and any combinations thereof.

In one embodiment of the composition for use according to the invention, the sortilin fragment, derivative or variant comprises the VPS10p, 10CCa, or 10CCb domain. Also included are sortilin fragments, derivatives or variants that include any combination of the above domains, for example, VPS10p+10CCa+10CCb domain, VPS10p+10CCa domain, VPS10p+10CCb domain or 10CCa+10CCb domain. The composition for use may comprise a dimer of any of the aforementioned polypeptides. By ‘dimer’ we intend a macromolecular complex formed by two single protein monomers, which may be expressed as a fusion protein.

The amino acid sequence of the Vps10p, 10CCa and 10CCb domain is provided herein as SEQ ID NO: 1, SEQ ID NO: 5, SEQ ID NO: 6, SEQ ID NO: 14 and SEQ ID NO: 18.

The amino acid sequence of the Vps10p+10CCa+10CCb domains, Vps10p+10CCa domains, Vps10p+10CCb domains and the 10CCa+10CCb domains are provided herein as SEQ ID NO: 4, SEQ ID NO: 2, SEQ ID NO: 3, SEQ ID NO: 7, SEQ ID NO: 17, SEQ ID NO: 15, SEQ ID NO: 16 and SEQ ID NO: 19.

The above amino acid sequences may or may not include protein tags. The presence of said tags may result in superior expression and/or an easier purification process. One such example could be the use of a HIS-tag, located either at the C-terminal (SEQ ID NO: 26) or at the N-terminal (SEQ ID NO: 27) with an additional TEV cleavage site. C-terminal HIS tagged sortilin is designed to have the wild type N-terminal after furin cleavage (allowing for protein activation). N-terminal HIS tagged sortilin is designed to have an N-terminal HIS tag after furin-mediated cleavage of the propeptide in the Golgi. The HIS tag can be cleaved off using a TEV protease, the latter of which recognises a strict 7 amino acid sequence of ENLYFQ(G/S) (SEQ ID NOs: 24 and 25, respectively).

The amino acid sequence of the C-terminal HIS tagged sortilin is herein provided as SEQ ID NO: 8 and SEQ ID NO: 20, and the amino acid sequence of the N-terminal HIS tagged sortilin is herein provided as SEQ ID NO: 10 and SEQ ID NO: 22.

It is intended that the above amino acid sequences may comprise an additional moiety and/or undergo a modification process. The skilled person would recognise that these modifications may result in improvements across a number of parameters, for example, enhanced stability, enhanced efficacy, improved half-life and/or reduced toxicity levels. Suitable additional moieties may include linker molecules, such as glutamic acid. These linker molecules may be of varying lengths, for example 2-31 amino acids in length. Lipophilic groups may also be attached. Examples of such lipophilic groups include fatty acids, isoprenoids, sterols, phospholipids and glycosylphosphatidyl inositol anchors. Possible modifications may include PEGylation, glycosylation or acetylation. PEGylation may involve the covalent or non-covalent addition of single, or multiple, polyethylene glycol polymer chains to the amino acid sequence. Glycosylation may involve the addition of a carbohydrate which is covalently attached to the amino acid sequence and acetylation refers to the process of introducing an acetyl group into said sequence. Further modifications may include methods to reduce binding of said sequence to proteoglycans or sulphur containing polysaccharides and oligosaccharides. Such modifications may utilise sulfatases to result in desulphation of said molecules.

The present invention provides any sortilin fragment or polypeptide with at least 70% homology to the aforementioned amino acid sequences. For example, the sortilin fragment or polypeptide of the invention may have at least 70%, at least 75%, at least 80%, at least 85%, at least 90%, at least 91%, at least 92%, at least 93%, at least 94%, at least 95%, at least 96%, at least 97%, at least 98% or at least 99% homology to the isolated polypeptides of any of SEQ ID NOs: 1, 2, 3, 4, 5, 6, 7, 8, 9, 10, 11, 14, 15, 16, 17, 18, 19, 20, 21, 22 and/or 23. Further, also included are polypeptides with one or more conservative substitutions. By ‘conservative substitution’ we intend an amino acid replacement that changes one or more amino acids whilst resulting in a substance with similar biochemical properties. These biochemical properties may include charge, hydrophobicity and size, as would be understood by a person of skill in the art.

In an embodiment, the fragment, derivative or variant of sortilin, or sortilin mimetic may bind to an insulin receptor. It is preferred that the fragment, derivative or variant of sortilin, or sortilin mimetic binds to a human insulin receptor. Alternatively, or additionally, the fragment, derivative or variant of sortilin, or sortilin mimetic may bind to an insulin receptor from any mammal, for example, a mouse, guinea pig, rat, rabbit, dog, cat, horse, cow, sheep or pig insulin receptor.

It is intended that the sortilin fragments, derivatives, variants or sortilin mimetics of the present invention interact with the insulin receptor, resulting in improved blood glucose level control. The present inventors have demonstrated that sortilin binds to the human/mouse insulin receptor in a dose-dependent manner with high affinity, displaying a Kd value of approximately 40 nM. The latter allows for the desired therapeutic effect (a reduction in blood glucose levels) of the sortilin fragments, derivative, variants or sortilin mimetics to occur at low concentrations, thus minimising the chances of associated adverse effects. The inventors also show that the presence of insulin does not affect the binding between sortilin and the insulin receptor, instead forming a trimeric receptor complex, thus allowing for continued insulin-driven cell signalling. Consequently, the use of subcutaneously administered human soluble sortilin is shown to significantly improve glucose handling after a glucose challenge in a diabetic mouse strain. This effect can be observed within less than 12 hours of treatment, displaying the impressive fast-acting properties of the compositions and polypeptides of the present invention.

In a second aspect, the invention provides an isolated polypeptide comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23, or an amino acid sequence having greater than 70% homology to said amino acid sequence, or a fragment thereof.

In a further aspect, the invention provides a nucleic acid encoding any of the polypeptides as defined herein.

The invention also provides a nucleic acid that hybridises to a nucleic acid of the invention under low and/or high stringency conditions. Accordingly, the nucleic acid may be complementary to the nucleic acid encoding a polypeptide of the invention. By “high stringency conditions” we include high temperature and low salt content in hybridisation buffers. By “low stringency conditions” we include lower temperatures and high salt content in hybridisation buffers. The present invention may involve the use of siRNAs (commonly referred to as small interfering or silencing RNA) or Morpholino oligomers, directed against the nucleotide sequences or regulatory portions thereof in vivo. By ‘siRNA’ we intend a class of double-stranded RNA molecules, commonly 20-25 base pairs in length. siRNAs operate within the RNA interference pathway and therefore interfere with gene expression. Most commonly, siRNAs prevent translation for the degradation of mRNA. By ‘Morpholino oligomers’ we intend an oligomer molecule wherein the structure has DNA bases attached to a backbone of methylenemorpholine rings, linked through phosphorodiamidate groups. These molecules commonly act via blocking the access of other molecules to specific sequences of the RNA molecule. The use of these molecules would be particularly relevant in the instance where the translation of the protein was inhibited, thus triggering a feedback loop wherein the transcript levels were in fact enhanced, leading to increased protein levels of sortilin fragments, derivatives or variants. Alternatively, these genetic tools could be targeted to transcripts known to be associated with protein regulation, in particular degradation, thus leading to an upregulation of sortilin protein.

In a further aspect, the present invention provides an expression vector comprising a nucleic acid according to the invention. The expression vector may be used to manufacture the polypeptides of the invention in vitro (for subsequent formulation and delivery) or indeed to be delivered direct to the patient to be treated where the expression of the polypeptides of the invention will occur in the patient themselves. Thus, the expression vector may be used in gene therapy to treat a condition identified herein.

The vector may be any suitable vector for ex vivo or in vivo expression. For example, the vector may a vector suitable for use in human gene therapy. The vector may alternatively be any vector suitable for use for expression in bacteria, mammalian cell lines, plants, yeasts, protozoa or cell free expression systems. Suitable examples of vectors according to the invention are viruses or plasmids. Examples of appropriate viruses include, but are not limited to, retroviruses, adenoviruses, adeno-associated viruses and the herpes simplex virus. Non-viral vector systems may also be used, for example, lipid DNA complexes. The promoter used to achieve activation of said nucleotide sequence may be selected from the group comprising: lentivirus, adeno-associated virus (AAV), cytomegalovirus (CMV), Rous sarcoma virus (RSV), elongation factor 1 alpha (EF1α), Tet-regulatable promoter, platelet-derived growth factor beta (PDGFβ), murine leukemia virus long terminal repeat (Mo-MLV-L TR), phosphoglycerate kinase (PGK), Mx1 and calmodulin-dependent protein kinase II (CaMK II). Ideally, the promoter will be tissue-specific to restrict unwanted transgene expression and to facilitate persistent transgene expression, preferentially in liver, adipose tissue or muscle. Liver specific promoters may include albumin (Palb), α1-antitrypsin (Pa1AT), hemopexin (Phpx), apolipoprotein A2 (APOA2), serpin peptidase inhibitor (SERPINA1), cytochrome p450 (CYP3A4), microRNA 122 (MIR122). Adipose tissue specific promoters may include adiponectin (AdipoQ) and muscle specific promoters may include muscle creatine kinase (MCK), myosin heavy chain 6 (Myh6), myosin light chain 2 (MYL2) and (tropomyosin 3) TPM3.

In a further aspect, the present invention provides a host cell or cell free expression system comprising an expression vector according to the invention. This may be for purposes of producing said polypeptide or for the purpose of advanced cell therapy techniques. The latter may involve isolation and manipulation of a patient's own cells to produce a desired alteration, before re-delivery to the patient. The host cell may be mammalian, bacterial, yeast, plant or insect derived. ‘Mammalian’ is taken to include cells from human, feline, porcine, simian, canine, murine and rat origin and may include the use of muscle cells, hepatocytes, adipocytes and cells of the pancreas, such as a, p and b cells. Conveniently Chinese hamster ovary (CHO) or Human embryonic kidney (HEK) cells may be utilised but any other suitable host cell may be used as would be understood by a person of skill in the art. Yeast systems which may be used include Pichia pastoris, Kluyveromyces lactis and Saccharomyces cerevisiae. Bacterial systems which would be appropriate for this purpose may include Escherichia coli, Corynebacterium, and Pseudomonas fluorescens. A cell free expression system may also be utilised wherein protein production is performed in vitro, using purified RNA polymerase, ribosomes, tRNA and ribonucleotides.

In a further aspect, the present invention provides a pharmaceutical composition comprising the isolated polypeptide or derivative or mimic, nucleic acid, expression vector or host cell of the preceding aspects and embodiments in combination with a pharmaceutically acceptable carrier or excipient. The excipients and carriers may enhance stability of the pharmaceutically active component, improve the biopharmaceutical profile and patient acceptability as well as simplify the manufacturing process, as would be understood by a person of skill in the art. Examples of suitable carriers or excipients include; sterile water, olive oil, ethyl oleate, glycols carbohydrates, especially monosaccharides such as fructose, glucose and galactose; non-reducing disaccharides such as sucrose, lactose and trehalose; non-reducing oligosaccharides such as raffinose and melezitose; non reducing starch derived polysaccharides products such as maltodextrins, dextrans and cyclodextrins; and non-reducing alditols such as mannitol and xylitol. Further suitable excipients include cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). Mixtures of two or more of any of the above excipients are also envisaged. Specifically, it is envisaged that simple saline solutions may be used for this purpose. Formulations that the present invention may take include lyophilised formulations and depot formulations, whereby the depot injection is given subcutaneously, intradermal or intramuscularly. Specific encapsulation techniques may be used as a drug delivery device, for example, PLGA (polylactic-co-glycolic acid) microspheres.

The present invention also provides the pharmaceutical composition according to the preceding aspect for use in medicine. In particular, the pharmaceutical composition for use herein may be for use in treating, or in the prevention of, diabetes mellitus, insulin resistance, obesity, metabolic disorders, polycystic ovary syndrome, non-alcoholic fatty liver disease, retinopathy, neuropathy, nephropathy, Alzheimer's disease and/or cardiovascular disease in a subject in need thereof.

Pharmaceutical compositions may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, pills, boluses, electuaries, or aerosols.

The pharmaceutical compositions may be prepared as suitable according to the intended route of administration. Possible routes of administration of said pharmaceutical composition include oral, sublingual, rectal, topical, parenteral and intravenous injection administration. Preferably, the chosen route of administration would be a subcutaneous injection, also known as SC, SQ, sub-cu, sub-Q or subcut. The site of injection may be the upper arm, the abdomen, the thigh, the upper back or the upper buttock.

The dose of the present invention is envisaged to be in the range of 0.01 to 1 mg/kg. The dose may be a daily dose of 0.05 mg/kg but ideally is administered once a week. The dosing regime may involve a single dose or multiple doses. It is understood that the exact dose and dosing regime will be dependent on the disease in question.

The present invention also provides for the use of a composition comprising a fragment, derivative or variant of sortilin, or a sortilin mimetic as defined herein, in the manufacture of a medicament for treating or preventing diabetes mellitus, insulin resistance, obesity, metabolic disorders, polycystic ovary syndrome, non-alcoholic fatty liver disease, retinopathy, neuropathy, nephropathy, Alzheimer's disease and/or cardiovascular disease in a subject in need thereof, wherein the sortilin mimetic is not SorCS1, SorCS2, SorCS3 or SorLA.

The invention further provides a method of treating or preventing a condition selected from diabetes mellitus, insulin resistance, obesity, metabolic disorders, polycystic ovary syndrome, non-alcoholic fatty liver disease, retinopathy, neuropathy, nephropathy, Alzheimer's disease and/or cardiovascular disease in a subject in need thereof, the method comprising administering a composition comprising a fragment, derivative or variant of sortilin, or a sortilin mimetic as defined herein to the subject.

The present invention also includes a method of manufacturing polypeptides or fragments thereof according to the invention comprising the step of expressing the nucleic acid of the invention in a host cell or other suitable expression system as indicated herein.

In order that the invention may be more clearly understood, embodiments thereof will now be described by way of example.

### EXAMPLES

### Example 1

The percentage level of identity between hSORT1 and hSORCS1 were investigated by aligning the protein sequences. Alignment software used by the inventors can be accessed via Uniprot.org.

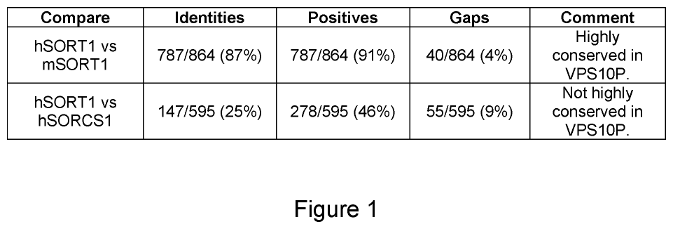

The data shows that between the human and murine forms of SORT1, there is a high level of homology (87%). However, when comparing the homology within the Vps10p domain between hSORT1 and hSORCS1, it was found to be substantially lower (25%) (FIG. 1).

### Example 2

Sortilin and insulin receptor binding was investigated using surface plasmon resonance analysis.

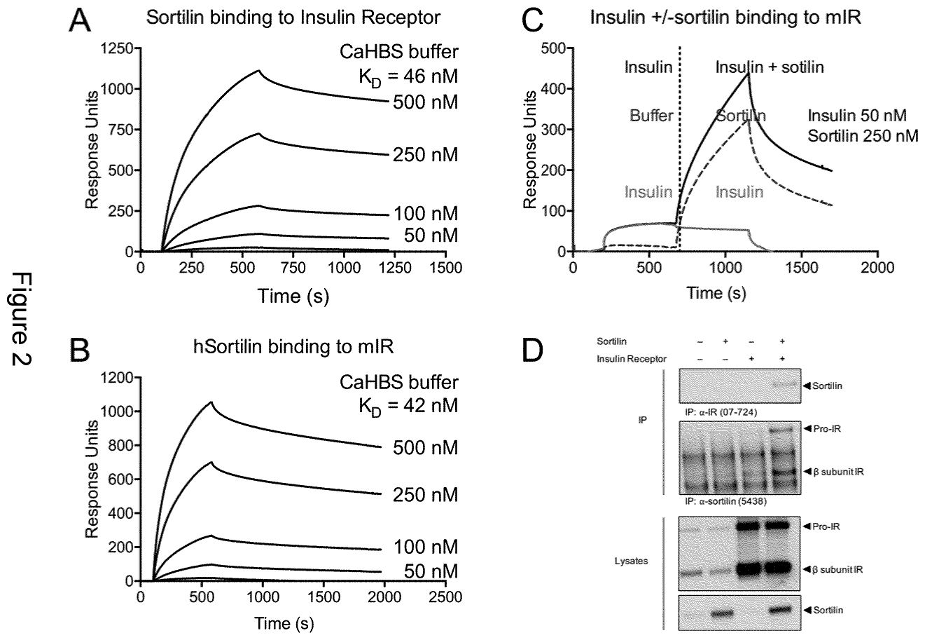

Soluble sortilin in concentrations ranging from 50-500 nM was assayed for binding to immobilized human insulin receptor (0.0609 pmol/mm²) and murine insulin receptor (0.0607 pmol/mm²) (FIGS. 2A and B). Sortilin showed dose-dependent binding to both the human and murine insulin receptor, with a Kd of approximately 40 nM. Insulin did not affect the binding between sortilin and the insulin receptor (FIG. 2C), indicating the formation of a trimeric receptor complex. Co-immunoprecipitation confirmed a physical interaction between sortilin and the human receptor with DSP crosslinker.

The data shows that sortilin and the insulin receptor are able to physically interact in a cell free system (Biacore). The interaction between sortilin and the insulin receptor does not impact on the binding of insulin to the insulin receptor.

### Example 3

RAP-purified soluble sortilin was tested in diabetic animal models (Db/Db) to investigate effects on plasma glucose levels, in steady state (basal fasting plasma glucose) and in dynamic state (IPGTT).

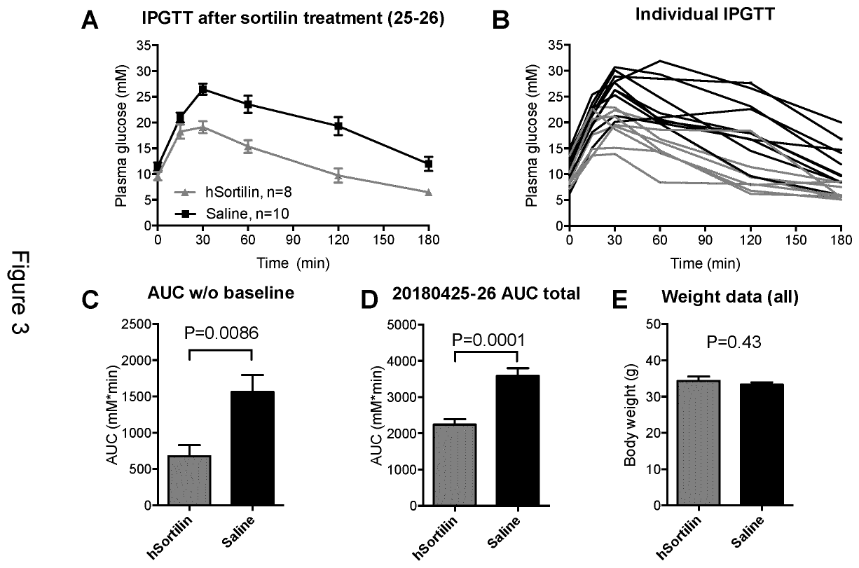

18 mice were ordered, aged 5 weeks at the point of delivery. The animals were allowed to acclimatise for 3 weeks prior to performing the experimental work. The animals were test fasted for 4 hrs and blood sampled 5 days before the experiment took place. The animals were randomised to either treatment or sham groups based on weight and plasma glucose levels. Following randomisation, the 18 db/db mice, now aged 8 weeks, were injected IP with 100 μg RAP-purified soluble sortilin (n=8) or saline (n=10) at 22:00 and at 08:00 the following morning. All animals were fasted from 22:00. The animals were subjected to an IPGTT at 09:00, with the dose of 1 mg/g BW in 200 mg/ml glucose (40 g=200 μL) (FIG. 3). The IPGTT curve showed a significant lower basal plasma glucose, and lower IPGTT AUC, both with and without correction for baseline plasma glucose (t=0 min).

### Example 4

The RAP-purified construct has a C-terminal HIS-tag which may engage with the heparin-sulfate proteoglycans to inhibit its activity. Therefore, this interaction and the effects on plasma glucose levels were investigated.

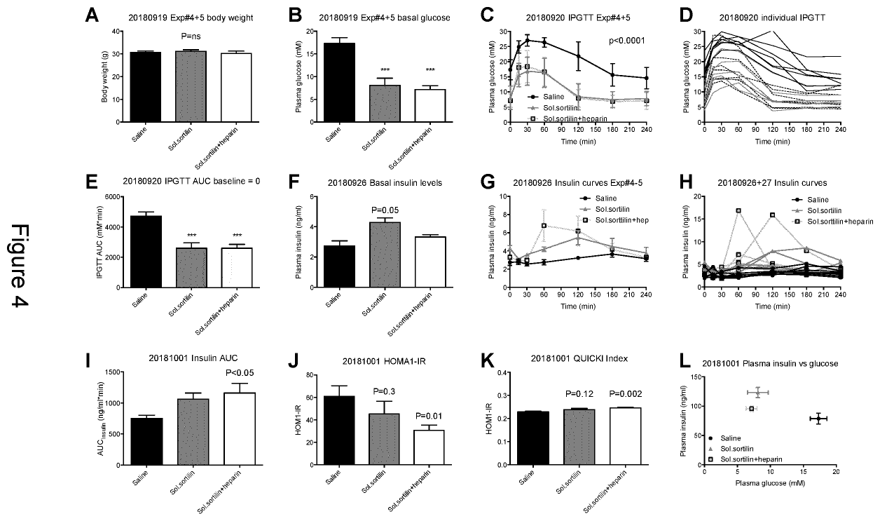

20 mice were ordered, aged 5 weeks at delivery. The animals were allowed to acclimatise for 2 weeks prior to performing the experimental work. The animals were test fasted for 4 hrs and sampled 5 days before the experiment took place. The animals were then randomised to either treatment or sham groups based on weight (approximately 30 g) and plasma glucose levels. The 20 db/db mice, now aged 7 weeks, were injected IP with 100 μg RAP-purified soluble sortilin and heparin (200 U/kg) (n=7) or without heparin (n=6), or saline (n=7) at 22:00 and at 08:00 the following morning. All animals were fasted from 22:00. The animals were subjected to an IPGTT at 09:00, with a dose of 1 mg/g BW in 200 mg/ml glucose (40 g=200 ul) (FIG. 4). The IPGTT curve showed a trend towards a lower basal plasma glucose, and lower IPGTT AUC, both total and baseline corrected. There was an effect of RAP-purified sortilin and no effect of added heparin. Insulin response after glucose challenge was improved when animals were treated with soluble sortilin (+/−heparin).

HOMA1-IR (resistance index) is calculated as (FPI×FPG)/405=HOMA1-IR, where FPI is fasting plasma insulin levels (mU/L), FPG is fasting plasma glucose (mg/dL) (Mather K. Am J Physiol Endocrinol Metab 296: E398-E399, 2009). QUICKI (sensitivity) Index is calculated as 1/(log(FPI mU/L)+log(FPG mg/dL) (Katz et al. JCEM 2000). Both QUICKI and HOMA1-IR indicates lower insulin resistance.

The data shows that RAP-purified soluble sortilin is active regardless of the presence of heparin during the glucose tolerance test.

| Sequences referenced throughout the specification and forming | |
|---|---|
| part of the description. | |
| (VPS10p Domain- isoform 1) | |
| SEQ ID NO: 1 | |
| 1 | RGGRWRRSAP GEDEECGRVR DFVAKLANNT HQHVFDDLRG SVSLSWVGDS |
| 51 | TGVILVLTTF HVPLVIMTFG QSKLYRSEDY GKNFKDITDL INNTFIRTEF |
| 101 | GMAIGPENSG KVVLTAEVSG GSRGGRIFRS SDFAKNFVQT DLPFHPLTQM |
| 151 | MYSPQNSDYL LALSTENGLW VSKNFGGKWE EIHKAVCLAK WGSDNTIFFT |
| 201 | TYANGSCKAD LGALELWRTS DLGKSFKTIG VKIYSFGLGG RFLFASVMAD |
| 251 | KDTTRRIHVS TDQGDTWSMA QLPSVGQEQF YSILAANDDM VFMHVDEPGD |
| 301 | TGFGTIFTSD DRGIVYSKSL DRHLYTTTGG ETDFTNVTSL RGVYITSVLS |
| 351 | EDNSIQTMIT FDQGGRWTHL RKPENSECDA TAKNKNECSL HIHASYSISQ |
| 401 | KLNVPMAPLS EPNAVGIVIA HGSVGDAISV MVPDVYISDD GGYSWTKMLE |
| 451 | GPHYYTILDS GGIIVAIEHS SRPINVIKFS TDEGQCWQTY TFTRDPIYFT |
| 501 | GLASEPGARS MNISIWGFTE SFLTSQWVSY TIDFKDILER |
| (VPS10p Domain + 10CCa Domain- isoform 1) | |
| SEQ ID NO: 2 | |
| 1 | RGGRWRRSAP GEDEECGRVR DFVAKLANNT HQHVFDDLRG SVSLSWVGDS |
| 51 | TGVILVLTTF HVPLVIMTFG QSKLYRSEDY GKNFKDITDL INNTFIRTEF |
| 101 | GMAIGPENSG KVVLTAEVSG GSRGGRIFRS SDFAKNFVQT DLPFHPLTQM |
| 151 | MYSPQNSDYL LALSTENGLW VSKNFGGKWE EIHKAVCLAK WGSDNTIFFT |
| 201 | TYANGSCKAD LGALELWRTS DLGKSFKTIG VKIYSFGLGG RFLFASVMAD |
| 251 | KDTTRRIHVS TDQGDTWSMA QLPSVGQEQF YSILAANDDM VFMHVDEPGD |
| 301 | TGFGTIFTSD DRGIVYSKSL DRHLYTTTGG ETDFTNVTSL RGVYITSVLS |
| 351 | EDNSIQTMIT FDQGGRWTHL RKPENSECDA TAKNKNECSL HIHASYSISQ |
| 401 | KLNVPMAPLS EPNAVGIVIA HGSVGDAISV MVPDVYISDD GGYSWTKMLE |
| 451 | GPHYYTILDS GGIIVAIEHS SRPINVIKFS TDEGQCWQTY TFTRDPIYFT |
| 501 | GLASEPGARS MNISIWGFTE SFLTSQWVSY TIDFKDILER NCEEKDYTIW |
| 551 | LAHSTDPEDY EDGCILGYKE QFLRLRKSSM CQNGRDYVVT KQPSICL |
| (VPS10p Domain + 10CCb Domain- isoform 1) | |
| SEQ ID NO: 3 | |
| 1 | RGGRWRRSAP GEDEECGRVR DFVAKLANNT HQHVFDDLRG SVSLSWVGDS |
| 51 | TGVILVLTTF HVPLVIMTFG QSKLYRSEDY GKNFKDITDL INNTFIRTEF |
| 101 | GMAIGPENSG KVVLTAEVSG GSRGGRIFRS SDFAKNFVQT DLPFHPLTQM |
| 151 | MYSPQNSDYL LALSTENGLW VSKNFGGKWE EIHKAVCLAK WGSDNTIFFT |
| 201 | TYANGSCKAD LGALELWRTS DLGKSFKTIG VKIYSFGLGG RFLFASVMAD |
| 251 | KDTTRRIHVS TDQGDTWSMA QLPSVGQEQF YSILAANDDM VFMHVDEPGD |
| 301 | TGFGTIFTSD DRGIVYSKSL DRHLYTTTGG ETDFTNVTSL RGVYITSVLS |
| 351 | EDNSIQTMIT FDQGGRWTHL RKPENSECDA TAKNKNECSL HIHASYSISQ |
| 401 | KLNVPMAPLS EPNAVGIVIA HGSVGDAISV MVPDVYISDD GGYSWTKMLE |
| 451 | GPHYYTILDS GGIIVAIEHS SRPINVIKFS TDEGQCWQTY TFTRDPIYFT |
| 501 | GLASEPGARS MNISIWGFTE SFLTSQWVSY TIDFKDILER CSLEDFLCDF |
| 551 | GYYRPENDSK CVEQPELKGH DLEFCLYGRE EHLTTNGYRK IPGDKCQGGV |
| 601 | NPVREVKDLK KKCTSNFLSP EKQ |
| (VPS10p Domain + 10CCa Domain + 10CCb Domain- isoform 1) | |
| SEQ ID NO: 4 | |
| 1 | RGGRWRRSAP GEDEECGRVR DFVAKLANNT HQHVFDDLRG SVSLSWVGDS |
| 51 | TGVILVLTTF HVPLVIMTFG QSKLYRSEDY GKNFKDITDL INNTFIRTEF |
| 101 | GMAIGPENSG KVVLTAEVSG GSRGGRIFRS SDFAKNFVQT DLPFHPLTQM |
| 151 | MYSPQNSDYL LALSTENGLW VSKNFGGKWE EIHKAVCLAK WGSDNTIFFT |
| 201 | TYANGSCKAD LGALELWRTS DLGKSFKTIG VKIYSFGLGG RFLFASVMAD |
| 251 | KDTTRRIHVS TDQGDTWSMA QLPSVGQEQF YSILAANDDM VFMHVDEPGD |
| 301 | TGFGTIFTSD DRGIVYSKSL DRHLYTTTGG ETDFTNVTSL RGVYITSVLS |
| 351 | EDNSIQTMIT FDQGGRWTHL RKPENSECDA TAKNKNECSL HIHASYSISQ |
| 401 | KLNVPMAPLS EPNAVGIVIA HGSVGDAISV MVPDVYISDD GGYSWTKMLE |
| 451 | GPHYYTILDS GGIIVAIEHS SRPINVIKFS TDEGQCWQTY TFTRDPIYFT |
| 501 | GLASEPGARS MNISIWGFTE SFLTSQWVSY TIDFKDILER NCEEKDYTIW |
| 551 | LAHSTDPEDY EDGCILGYKE QFLRLRKSSM CQNGRDYVVT KQPSICLCSL |
| 601 | EDFLCDFGYY RPENDSKCVE QPELKGHDLE FCLYGREEHL TTNGYRKIPG |
| 651 | DKCQGGVNPV REVKDLKKKC TSNFLSPEKQ |
| (10CCa Domain- isoform 1) | |
| SEQ ID NO: 5 | |
| 1 | NCEEKDYTIW LAHSTDPEDY EDGCILGYKE QFLRLRKSSM CQNGRDYVVT |
| 51 | KQPSICL |
| (10CCb Domain- isoform 1) | |
| SEQ ID NO: 6 | |
| 1 | CSLEDFLCDF GYYRPENDSK CVEQPELKGH DLEFCLYGRE EHLTTNGYRK |
| 51 | IPGDKCQGGV NPVREVKDLK KKCTSNFLSP EKQ |
| (10CCa + 10CCb Domain- isoform 1) | |
| SEQ ID NO: 7 | |
| 1 | NCEEKDYTIW LAHSTDPEDY EDGCILGYKE QFLRLRKSSM CQNGRDYVVT |
| 51 | KQPSICLCSL EDFLCDFGYY RPENDSKCVE QPELKGHDLE FCLYGREEHL |
| 101 | TTNGYRKIPG DKCQGGVNPV REVKDLKKKC TSNFLSPEKQ |
| (IST218 after furin site plus HIS tag- isoform 1) | |
| SEQ ID NO: 8 | |
| 1 | SAPGEDEECG RVRDFVAKLA NNTHQHVFDD LRGSVSLSWV GDSTGVILVL |
| 51 | TTFHVPLVIM TFGQSKLYRS EDYGKNFKDI TDLINNTFIR TEFGMAIGPE |
| 101 | NSGKVVLTAE VSGGSRGGRI FRSSDFAKNF VQTDLPFHPL TQMMYSPQNS |
| 151 | DYLLALSTEN GLWVSKNFGG KWEEIHKAVC LAKWGSDNTI FFTTYANGSC |
| 201 | KADLGALELW RTSDLGKSFK TIGVKIYSFG LGGRFLFASV MADKDTTRRI |
| 251 | HVSTDQGDTW SMAQLPSVGQ EQFYSILAAN DDMVFMHVDE PGDTGFGTIF |
| 301 | TSDDRGIVYS KSLDRHLYTT TGGETDFTNV TSLRGVYITS VLSEDNSIQT |
| 351 | MITFDQGGRW THLRKPENSE CDATAKNKNE CSLHIHASYS ISQKLNVPMA |
| 401 | PLSEPNAVGI VIAHGSVGDA ISVMVPDVYI SDDGGYSWTK MLEGPHYYTI |
| 451 | LDSGGIIVAI EHSSRPINVI KFSTDEGQCW QTYTFTRDPI YFTGLASEPG |
| 501 | ARSMNISIWG FTESFLTSQW VSYTIDFKDI LERNCEEKDY TIWLAHSTDP |
| 551 | EDYEDGCILG YKEQFLRLRK SSMCQNGRDY VVTKQPSICL CSLEDFLCDF |
| 601 | GYYRPENDSK CVEQPELKGH DLEFCLYGRE EHLTTNGYRK IPGDKCQGGV |
| 651 | NPVREVKDLK KKCTSNFLSP EKQNSKSNSH HHHHH |
| (IST218 after furin site minus HIS tag- isoform 1) | |
| SEQ ID NO: 9 | |
| 1 | SAPGEDEECG RVRDFVAKLA NNTHQHVFDD LRGSVSLSWV GDSTGVILVL |
| 51 | TTFHVPLVIM TFGQSKLYRS EDYGKNFKDI TDLINNTFIR TEFGMAIGPE |
| 101 | NSGKVVLTAE VSGGSRGGRI FRSSDFAKNF VQTDLPFHPL TQMMYSPQNS |
| 151 | DYLLALSTEN GLWVSKNFGG KWEEIHKAVC LAKWGSDNTI FFTTYANGSC |
| 201 | KADLGALELW RTSDLGKSFK TIGVKIYSFG LGGRFLFASV MADKDTTRRI |
| 251 | HVSTDQGDTW SMAQLPSVGQ EQFYSILAAN DDMVFMHVDE PGDTGFGTIF |
| 301 | TSDDRGIVYS KSLDRHLYTT TGGETDFTNV TSLRGVYITS VLSEDNSIQT |
| 351 | MITFDQGGRW THLRKPENSE CDATAKNKNE CSLHIHASYS ISQKLNVPMA |
| 401 | PLSEPNAVGI VIAHGSVGDA ISVMVPDVYI SDDGGYSWTK MLEGPHYYTI |
| 451 | LDSGGIIVAI EHSSRPINVI KFSTDEGQCW QTYTFTRDPI YFTGLASEPG |
| 501 | ARSMNISIWG FTESFLTSQW VSYTIDFKDI LERNCEEKDY TIWLAHSTDP |
| 551 | EDYEDGCILG YKEQFLRLRK SSMCQNGRDY VVTKQPSICL CSLEDFLCDF |
| 601 | GYYRPENDSK CVEQPELKGH DLEFCLYGRE EHLTTNGYRK IPGDKCQGGV |
| 651 | NPVREVKDLK KKCTSNFLSP EKQNSKSNS |
| (SKT1 after furin site plus HIS tag + TEV site- isoform 1) | |
| SEQ ID NO: 10 | |
| 1 | SAPGHHHHHH HENLYFQSED EECGRVRDFV AKLANNTHQH VFDDLRGSVS |
| 51 | LSWVGDSTGV ILVLTTFHVP LVIMTFGQSK LYRSEDYGKN FKDITDLINN |
| 101 | TFIRTEFGMA IGPENSGKVV LTAEVSGGSR GGRIFRSSDF AKNFVQTDLP |
| 151 | FHPLTQMMYS PQNSDYLLAL STENGLWVSK NFGGKWEEIH KAVCLAKWGS |
| 201 | DNTIFFTTYA NGSCKADLGA LELWRTSDLG KSFKTIGVKI YSFGLGGRFL |
| 251 | FASVMADKDT TRRIHVSTDQ GDTWSMAQLP SVGQEQFYSI LAANDDMVFM |
| 301 | HVDEPGDTGF GTIFTSDDRG IVYSKSLDRH LYTTTGGETD FTNVTSLRGV |
| 351 | YITSVLSEDN SIQTMITFDQ GGRWTHLRKP ENSECDATAK NKNECSLHIH |
| 401 | ASYSISQKLN VPMAPLSEPN AVGIVIAHGS VGDAISVMVP DVYISDDGGY |
| 451 | SWTKMLEGPH YYTILDSGGI IVAIEHSSRP INVIKFSTDE GQCWQTYTFT |
| 501 | RDPIYFTGLA SEPGARSMNI SIWGFTESFL TSQWVSYTID FKDILERNCE |
| 551 | EKDYTIWLAH STDPEDYEDG CILGYKEQFL RLRKSSMCQN GRDYVVTKQP |
| 601 | SICLCSLEDF LCDFGYYRPE NDSKCVEQPE LKGHDLEFCL YGREEHLTTN |
| 651 | GYRKIPGDKC QGGVNPVREV KDLKKKCTSN |
| (SKT1 after furin site minus HIS tag + TEV site- isoform 1) | |
| SEQ ID NO: 11 | |
| 1 | EDEECGRVRD FVAKLANNTH QHVFDDLRGS VSLSWVGDST GVILVLTTFH |
| 51 | VPLVIMTFGQ SKLYRSEDYG KNFKDITDLI NNTFIRTEFG MAIGPENSGK |
| 101 | VVLTAEVSGG SRGGRIFRSS DFAKNFVQTD LPFHPLTQMM YSPQNSDYLL |
| 151 | ALSTENGLWV SKNFGGKWEE IHKAVCLAKW GSDNTIFFTT YANGSCKADL |
| 201 | GALELWRTSD LGKSFKTIGV KIYSFGLGGR FLFASVMADK DTTRRIHVST |
| 251 | DQGDTWSMAQ LPSVGQEQFY SILAANDDMV FMHVDEPGDT GFGTIFTSDD |
| 301 | RGIVYSKSLD RHLYTTTGGE TDFTNVTSLR GVYITSVLSE DNSIQTMITF |
| 351 | DQGGRWTHLR KPENSECDAT AKNKNECSLH IHASYSISQK LNVPMAPLSE |
| 401 | PNAVGIVIAH GSVGDAISVM VPDVYISDDG GYSWTKMLEG PHYYTILDSG |
| 451 | GIIVAIEHSS RPINVIKFST DEGQCWQTYT FTRDPIYFTG LASEPGARSM |
| 501 | NISIWGFTES FLTSQWVSYT IDFKDILERN CEEKDYTIWL AHSTDPEDYE |
| 551 | DGCILGYKEQ FLRLRKSSMC QNGRDYVVTK QPSICLCSLE DFLCDFGYYR |
| 601 | PENDSKCVEQ PELKGHDLEF CLYGREEHLT TNGYRKIPGD KCQGGVNPVR |
| 651 | EVKDLKKKCT SN |
| (full length sortilin- isoform 1) | |
| SEQ ID NO: 12 | |
| 1 | MERPWGAADG LSRWPHGLGL LLLLQLLPPS TLSQDRLDAP PPPAAPLPRW |
| 51 | SGPIGVSWGL RAAAAGGAFP RGGRWRRSAP GEDEECGRVR DFVAKLANNT |
| 101 | HQHVFDDLRG SVSLSWVGDS TGVILVLTTF HVPLVIMTFG QSKLYRSEDY |
| 151 | GKNFKDITDL INNTFIRTEF GMAIGPENSG KVVLTAEVSG GSRGGRIFRS |
| 201 | SDFAKNFVQT DLPFHPLTQM MYSPQNSDYL LALSTENGLW VSKNFGGKWE |
| 251 | EIHKAVCLAK WGSDNTIFFT TYANGSCKAD LGALELWRTS DLGKSFKTIG |
| 301 | VKIYSFGLGG RFLFASVMAD KDTTRRIHVS TDQGDTWSMA QLPSVGQEQF |
| 351 | YSILAANDDM VFMHVDEPGD TGFGTIFTSD DRGIVYSKSL DRHLYTTTGG |
| 401 | ETDFTNVTSL RGVYITSVLS EDNSIQTMIT FDQGGRWTHL RKPENSECDA |
| 451 | TAKNKNECSL HIHASYSISQ KLNVPMAPLS EPNAVGIVIA HGSVGDAISV |
| 501 | MVPDVYISDD GGYSWTKMLE GPHYYTILDS GGIIVAIEHS SRPINVIKFS |
| 551 | TDEGQCWQTY TFTRDPIYFT GLASEPGARS MNISIWGFTE SFLTSQWVSY |
| 601 | TIDFKDILER NCEEKDYTIW LAHSTDPEDY EDGCILGYKE QFLRLRKSSM |
| 651 | CQNGRDYVVT KQPSICLCSL EDFLCDFGYY RPENDSKCVE QPELKGHDLE |
| 701 | FCLYGREEHL TTNGYRKIPG DKCQGGVNPV REVKDLKKKC TSNFLSPEKQ |
| 751 | NSKSNSVPII LAIVGLMLVT VVAGVLIVKK YVCGGRFLVH RYSVLQQHAE |
| 801 | ANGVDGVDAL DTASHTNKSG YHDDSDEDLL E |
| (full length sortilin- isoform 2) | |
| SEQ ID NO: 13 | |
| 1 | MERPWGAADG LSRWPHGLGL LLLLQLLPPS TLSQDRLDAP PPPAAPLPRW |
| 51 | SGPIGVSWGL RAAAAGGAFP RGGRWRRSAP GEDEECGRVR DFVAKLANNT |
| 101 | HQHVFDDLRG SVSLSWVGDS TGVILVLTTF HVPLVIMTFG QSKLYRSEDY |
| 151 | GKNFKDITDL INNTFIRTEF GMAIGPENSG KVVLTAEVSG GSRGGRIFRS |
| 201 | SDFAKNFVQT DLPFHPLTQM MYSPQNSDYL LALSTENGLW VSKNFGGKWE |
| 251 | EIHKAVCLAK WGSDNTIFFT TYANGSCTDL GALELWRTSD LGKSFKTIGV |
| 301 | KIYSFGLGGR FLFASVMADK DTTRRIHVST DQGDTWSMAQ LPSVGQEQFY |
| 351 | SILAANDDMV FMHVDEPGDT GFGTIFTSDD RGIVYSKSLD RHLYTTTGGE |
| 401 | TDFTNVTSLR GVYITSVLSE DNSIQTMITF DQGGRWTHLR KPENSECDAT |
| 451 | AKNKNECSLH IHASYSISQK LNVPMAPLSE PNAVGIVIAH GSVGDAISVM |
| 501 | VPDVYISDDG GYSWTKMLEG PHYYTILDSG GIIVAIEHSS RPINVIKFST |
| 551 | DEGQCWQTYT FTRDPIYFTG LASEPGARSM NISIWGFTES FLTSQWVSYT |
| 601 | IDFKDILERN CEEKDYTIWL AHSTDPEDYE DGCILGYKEQ FLRLRKSSVC |
| 651 | QNGRDYVVTK QPSICLCSLE DFLCDFGYYR PENDSKCVEQ PELKGHDLEF |
| 701 | CLYGREEHLT TNGYRKIPGD KCQGGVNPVR EVKDLKKKCT SNFLSPEKQN |
| 751 | SKSNSVPIIL AIVGLMLVTV VAGVLIVKKY VCGGRFLVHR YSVLQQHAEA |
| 801 | NGVDGVDALD TASHTNKSGY HDDSDEDLLE |
| (VPS10p Domain- isoform 2) | |
| SEQ ID NO: 14 | |
| 1 | RGGRWRRSAP GEDEECGRVR DFVAKLANNT HQHVFDDLRG SVSLSWVGDS |
| 51 | TGVILVLTTF HVPLVIMTFG QSKLYRSEDY GKNFKDITDL INNTFIRTEF |
| 101 | GMAIGPENSG KVVLTAEVSG GSRGGRIFRS SDFAKNFVQT DLPFHPLTQM |
| 151 | MYSPQNSDYL LALSTENGLW VSKNFGGKWE EIHKAVCLAK WGSDNTIFFT |
| 201 | TYANGSCTDL GALELWRTSD LGKSFKTIGV KIYSFGLGGR FLFASVMADK |
| 251 | DTTRRIHVST DQGDTWSMAQ LPSVGQEQFY SILAANDDMV FMHVDEPGDT |
| 300 | GFGTIFTSDD RGIVYSKSLD RHLYTTTGGE TDFTNVTSLR GVYITSVLSE |
| 351 | DNSIQTMITF DQGGRWTHLR KPENSECDAT AKNKNECSLH IHASYSISQK |
| 400 | LNVPMAPLSE PNAVGIVIAH GSVGDAISVM VPDVYISDDG GYSWTKMLEG |
| 401 | PHYYTILDSG GIIVAIEHSS RPINVIKFST DEGQCWQTYT FTRDPIYFTG |
| 451 | LASEPGARSM NISIWGFTES FLTSQWVSYT IDFKDILER |
| (VPS10p Domain + 10CCa Domain- isoform 2) | |
| SEQ ID NO: 15 | |
| 1 | RGGRWRRSAP GEDEECGRVR DFVAKLANNT HQHVFDDLRG SVSLSWVGDS |
| 51 | TGVILVLTTF HVPLVIMTFG QSKLYRSEDY GKNFKDITDL INNTFIRTEF |
| 101 | GMAIGPENSG KVVLTAEVSG GSRGGRIFRS SDFAKNFVQT DLPFHPLTQM |
| 151 | MYSPQNSDYL LALSTENGLW VSKNFGGKWE EIHKAVCLAK WGSDNTIFFT |
| 201 | TYANGSCTDL GALELWRTSD LGKSFKTIGV KIYSFGLGGR FLFASVMADK |
| 251 | DTTRRIHVST DQGDTWSMAQ LPSVGQEQFY SILAANDDMV FMHVDEPGDT |
| 300 | GFGTIFTSDD RGIVYSKSLD RHLYTTTGGE TDFTNVTSLR GVYITSVLSE |
| 351 | DNSIQTMITF DQGGRWTHLR KPENSECDAT AKNKNECSLH IHASYSISQK |
| 400 | LNVPMAPLSE PNAVGIVIAH GSVGDAISVM VPDVYISDDG GYSWTKMLEG |
| 401 | PHYYTILDSG GIIVAIEHSS RPINVIKFST DEGQCWQTYT FTRDPIYFTG |
| 451 | LASEPGARSM NISIWGFTES FLTSQWVSYT IDFKDILERN CEEKDYTIWL |
| 501 | AHSTDPEDYE DGCILGYKEQ FLRLRKSSVC QNGRDYVVTK QPSICL |
| (VPS10p Domain + 10CCb Domain- isoform 2) | |
| SEQ ID NO: 16 | |
| 1 | RGGRWRRSAP GEDEECGRVR DFVAKLANNT HQHVFDDLRG SVSLSWVGDS |
| 51 | TGVILVLTTF HVPLVIMTFG QSKLYRSEDY GKNFKDITDL INNTFIRTEF |
| 101 | GMAIGPENSG KVVLTAEVSG GSRGGRIFRS SDFAKNFVQT DLPFHPLTQM |
| 151 | MYSPQNSDYL LALSTENGLW VSKNFGGKWE EIHKAVCLAK WGSDNTIFFT |
| 201 | TYANGSCTDL GALELWRTSD LGKSFKTIGV KIYSFGLGGR FLFASVMADK |
| 251 | DTTRRIHVST DQGDTWSMAQ LPSVGQEQFY SILAANDDMV FMHVDEPGDT |
| 300 | GFGTIFTSDD RGIVYSKSLD RHLYTTTGGE TDFTNVTSLR GVYITSVLSE |
| 351 | DNSIQTMITF DQGGRWTHLR KPENSECDAT AKNKNECSLH IHASYSISQK |
| 400 | LNVPMAPLSE PNAVGIVIAH GSVGDAISVM VPDVYISDDG GYSWTKMLEG |
| 401 | PHYYTILDSG GIIVAIEHSS RPINVIKFST DEGQCWQTYT FTRDPIYFTG |
| 451 | LASEPGARSM NISIWGFTES FLTSQWVSYT IDFKDILERC SLEDFLCDFG |
| 501 | YYRPENDSKC VEQPELKGHD LEFCLYGREE HLTTNGYRKI PGDKCQGGVN |
| 551 | PVREVKDLKK KCTSNFLSPE KQ |
| (VPS10p Domain + 10CCa Domain + 10CCb Domain- isoform 2) | |
| SEQ ID NO: 17 | |
| 1 | RGGRWRRSAP GEDEECGRVR DFVAKLANNT HQHVFDDLRG SVSLSWVGDS |
| 51 | TGVILVLTTF HVPLVIMTFG QSKLYRSEDY GKNFKDITDL INNTFIRTEF |
| 101 | GMAIGPENSG KVVLTAEVSG GSRGGRIFRS SDFAKNFVQT DLPFHPLTQM |
| 151 | MYSPQNSDYL LALSTENGLW VSKNFGGKWE EIHKAVCLAK WGSDNTIFFT |
| 201 | TYANGSCTDL GALELWRTSD LGKSFKTIGV KIYSFGLGGR FLFASVMADK |
| 251 | DTTRRIHVST DQGDTWSMAQ LPSVGQEQFY SILAANDDMV FMHVDEPGDT |
| 300 | GFGTIFTSDD RGIVYSKSLD RHLYTTTGGE TDFTNVTSLR GVYITSVLSE |
| 351 | DNSIQTMITF DQGGRWTHLR KPENSECDAT AKNKNECSLH IHASYSISQK |
| 400 | LNVPMAPLSE PNAVGIVIAH GSVGDAISVM VPDVYISDDG GYSWTKMLEG |
| 401 | PHYYTILDSG GIIVAIEHSS RPINVIKFST DEGQCWQTYT FTRDPIYFTG |
| 451 | LASEPGARSM NISIWGFTES FLTSQWVSYT IDFKDILERN CEEKDYTIWL |
| 501 | AHSTDPEDYE DGCILGYKEQ FLRLRKSSVC QNGRDYVVTK QPSICLCSLE |
| 551 | DFLCDFGYYR PENDSKCVEQ PELKGHDLEF CLYGREEHLT TNGYRKIPGD |
| 601 | KCQGGVNPVR EVKDLKKKCT SNFLSPEKQ |
| (10CCa Domain- isoform 2) | |
| SEQ ID NO: 18 | |
| 1 | NCEEKDYTIW LAHSTDPEDY EDGCILGYKE QFLRLRKSSV CQNGRDYVVT |
| 51 | KQPSICL |
| (10CCa Domain + 10CCb Domain- isoform 2) | |
| SEQ ID NO: 19 | |
| 1 | NCEEKDYTIW LAHSTDPEDY EDGCILGYKE QFLRLRKSSV CQNGRDYVVT |
| 51 | KQPSICLCSL EDFLCDFGYY RPENDSKCVE QPELKGHDLE FCLYGREEHL |
| 101 | TTNGYRKIPG DKCQGGVNPV REVKDLKKKC TSNFLSPEKQ |
| (IST218 after furin site plus HIS tag- isoform 2) | |
| SEQ ID NO: 20 | |
| 1 | SAPGEDEECG RVRDFVAKLA NNTHQHVFDD LRGSVSLSWV GDSTGVILVL |
| 51 | TTFHVPLVIM TFGQSKLYRS EDYGKNFKDI TDLINNTFIR TEFGMAIGPE |
| 101 | NSGKVVLTAE VSGGSRGGRI FRSSDFAKNF VQTDLPFHPL TQMMYSPQNS |
| 151 | DYLLALSTEN GLWVSKNFGG KWEEIHKAVC LAKWGSDNTI FFTTYANGSC |
| 201 | TDLGALELWR TSDLGKSFKT IGVKIYSFGL GGRFLFASVM ADKDTTRRIH |
| 251 | VSTDQGDTWS MAQLPSVGQE QFYSILAAND DMVFMHVDEP GDTGFGTIFT |
| 301 | SDDRGIVYSK SLDRHLYTTT GGETDFTNVT SLRGVYITSV LSEDNSIQTM |
| 351 | ITFDQGGRWT HLRKPENSEC DATAKNKNEC SLHIHASYSI SQKLNVPMAP |
| 401 | LSEPNAVGIV IANGSVGDAI SVMVPDVYIS DDGGYSWTKM LEGPHYYTIL |
| 451 | DSGGIIVAIE HSSRPINVIK FSTDEGQCWQ TYTFTRDPIY FTGLASEPGA |
| 501 | RSMNISIWGF TESFLTSQWV SYTIDFKDIL ERNCEEKDYT IWLAHSTDPE |
| 551 | DYEDGCILGY KEQFLRLRKS SVCQNGRDYV VTKQPSICLC SLEDFLCDFG |
| 601 | YYRPENDSKC VEQPELKGHD LEFCLYGREE HLTTNGYRKI PGDKCQGGVN |
| 651 | PVREVKDLKK KCTSNFLSPE KQNSKSNSHH HHHH |
| (IST218 after furin site minus HIS tag- isoform 2) | |
| SEQ ID NO: 21 | |
| 1 | SAPGEDEECG RVRDFVAKLA NNTHQHVFDD LRGSVSLSWV GDSTGVILVL |
| 51 | TTFHVPLVIM TFGQSKLYRS EDYGKNFKDI TDLINNTFIR TEFGMAIGPE |
| 101 | NSGKVVLTAE VSGGSRGGRI FRSSDFAKNF VQTDLPFHPL TQMMYSPQNS |
| 151 | DYLLALSTEN GLWVSKNFGG KWEEIHKAVC LAKWGSDNTI FFTTYANGSC |
| 201 | TDLGALELWR TSDLGKSFKT IGVKIYSFGL GGRFLFASVM ADKDTTRRIH |
| 251 | VSTDQGDTWS MAQLPSVGQE QFYSILAAND DMVFMHVDEP GDTGFGTIFT |
| 301 | SDDRGIVYSK SLDRHLYTTT GGETDFTNVT SLRGVYITSV LSEDNSIQTM |
| 351 | ITFDQGGRWT HLRKPENSEC DATAKNKNEC SLHIHASYSI SQKLNVPMAP |
| 401 | LSEPNAVGIV IANGSVGDAI SVMVPDVYIS DDGGYSWTKM LEGPHYYTIL |
| 451 | DSGGIIVAIE HSSRPINVIK FSTDEGQCWQ TYTFTRDPIY FTGLASEPGA |
| 501 | RSMNISIWGF TESFLTSQWV SYTIDFKDIL ERNCEEKDYT IWLAHSTDPE |
| 551 | DYEDGCILGY KEQFLRLRKS SVCQNGRDYV VTKQPSICLC SLEDFLCDFG |
| 601 | YYRPENDSKC VEQPELKGHD LEFCLYGREE HLTTNGYRKI PGDKCQGGVN |
| 651 | PVREVKDLKK KCTSNFLSPE KQNSKSNS |
| (SKT1 after furin site plus HIS tag + TEV site- isoform 2) | |
| SEQ ID NO: 22 | |
| 1 | SAPGHHHHHH HENLYFQSED EECGRVRDFV AKLANNTHQH VFDDLRGSVS |
| 51 | LSWVGDSTGV ILVLTTFHVP LVIMTFGQSK LYRSEDYGKN FKDITDLINN |
| 101 | TFIRTEFGMA IGPENSGKVV LTAEVSGGSR GGRIFRSSDF AKNFVQTDLP |
| 151 | FHPLTQMMYS PQNSDYLLAL STENGLWVSK NFGGKWEEIH KAVCLAKWGS |
| 201 | DNTIFFTTYA NGSCTDLGAL ELWRTSDLGK SFKTIGVKIY SFGLGGRFLF |
| 251 | ASVMADKDTT RRIHVSTDQG DTWSMAQLPS VGQEQFYSIL AANDDMVFMH |
| 301 | VDEPGDTGFG TIFTSDDRGI VYSKSLDRHL YTTTGGETDF TNVTSLRGVY |
| 351 | ITSVLSEDNS IQTMITFDQG GRWTHLRKPE NSECDATAKN KNECSLHIHA |
| 401 | SYSISQKLNV PMAPLSEPNA VGIVIAHGSV GDAISVMVPD VYISDDGGYS |
| 451 | WTKMLEGPHY YTILDSGGII VAIEHSSRPI NVIKFSTDEG QCWQTYTFTR |
| 501 | DPIYFTGLAS EPGARSMNIS IWGFTESFLT SQWVSYTIDF KDILERNCEE |
| 551 | KDYTIWLAHS TDPEDYEDGC ILGYKEQFLR LRKSSVCQNG RDYVVTKQPS |
| 601 | ICLCSLEDFL CDFGYYRPEN DSKCVEQPEL KGHDLEFCLY GREEHLTTNG |
| 651 | YRKIPGDKCQ GGVNPVREVK DLKKKCTSN |
| (SKT1 after furin site minus HIS tag + TEV site- isoform 2) | |
| SEQ ID NO: 23 | |
| 1 | EDEECGRVRD FVAKLANNTH QHVFDDLRGS VSLSWVGDST GVILVLTTFH |
| 51 | VPLVIMTFGQ SKLYRSEDYG KNFKDITDLI NNTFIRTEFG MAIGPENSGK |
| 101 | VVLTAEVSGG SRGGRIFRSS DFAKNFVQTD LPFHPLTQMM YSPQNSDYLL |
| 151 | ALSTENGLWV SKNFGGKWEE IHKAVCLAKW GSDNTIFFTT YANGSCTDLG |
| 201 | ALELWRTSDL GKSFKTIGVK IYSFGLGGRF LFASVMADKD TTRRIHVSTD |
| 251 | QGDTWSMAQL PSVGQEQFYS ILAANDDMVF MHVDEPGDTG FGTIFTSDDR |
| 301 | GIVYSKSLDR HLYTTTGGET DFTNVTSLRG VYITSVLSED NSIQTMITFD |
| 351 | QGGRWTHLRK PENSECDATA KNKNECSLHI HASYSISQKL NVPMAPLSEP |
| 401 | NAVGIVIAHG SVGDAISVMV PDVYISDDGG YSWTKMLEGP HYYTILDSGG |
| 451 | IIVAIEHSSR PINVIKFSTD EGQCWQTYTF TRDPIYFTGL ASEPGARSMN |
| 501 | ISIWGFTESF LTSQWVSYTI DFKDILERNC EEKDYTIWLA HSTDPEDYED |
| 551 | GCILGYKEQF LRLRKSSVCQ NGRDYVVTKQ PSICLCSLED FLCDFGYYRP |
| 601 | ENDSKCVEQP ELKGHDLEFC LYGREEHLTT NGYRKIPGDK CQGGVNPVRE |
| 651 | VKDLKKKCTS N |
| (TEV cleavage site option 1) | |
| SEQ ID NO: 24 | |
| 1 | ENLYFQG |
| (TEV cleavage site option 2) | |
| SEQ ID NO: 25 | |
| 1 | ENLYFQS |
| (C terminal HIS tag) | |
| SEQ ID NO: 26 | |
| 1 | HHHHHH |
| (N terminal HIS tag) | |
| SEQ ID NO: 27 | |
| 1 | HHHHHHH |

1.-15. (canceled)

16. A method of treating or preventing diabetes mellitus, insulin resistance, obesity, metabolic disorders, polycystic ovary syndrome, non-alcoholic fatty liver disease, retinopathy, neuropathy, nephropathy, Alzheimer's disease or cardiovascular disease in a subject in need thereof comprising administering a composition comprising a fragment, derivative or variant of sortilin, or a sortilin mimetic, wherein the sortilin mimetic is not SorCS1, SorCS2, SorCS3 or SorLA.

17. The method according to claim 16, wherein the sortilin mimetic is an antibody, nanobody or aptamer.

18. The method according to claim 16, wherein the fragment, derivative or variant comprises a VPS10p, 10CCa or 10CCb domain.

19. The method according to claim 16, wherein the fragment, derivative or variant of sortilin comprises the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23, or an amino acid sequence having greater than 70% homology to the amino acid sequence.

20. The method according to claim 16, wherein the fragment, derivative or variant of sortilin, or sortilin mimetic, comprises an additional moiety or a modification.

21. The method according to claim 20, wherein the additional moiety is a linker molecule or wherein the modification is PEGylation, glycosylation or acetylation.

22. The method according to claim 16, wherein the fragment, derivative or variant of sortilin, or sortilin mimetic binds to an insulin receptor.

23. The method according to claim 22, wherein the insulin receptor is a human or murine insulin receptor.

24. A method of treating or preventing diabetes mellitus, insulin resistance, obesity, metabolic disorders, polycystic ovary syndrome, non-alcoholic fatty liver disease, retinopathy, neuropathy, nephropathy, Alzheimer's disease and/or cardiovascular disease in a subject in need thereof comprising administering the composition of claim 16.

25. An isolated polypeptide comprising the amino acid sequence of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22 or SEQ ID NO: 23, or an amino acid sequence having greater than 70% homology to the amino acid sequence, or a fragment thereof.

26. A nucleic acid encoding a polypeptide according to claim 25.

27. A nucleic acid that hybridises to a nucleic acid according to claim 26.

28. An expression vector comprising a nucleic acid according to claim 26.

29. An expression vector comprising a nucleic acid according to claim 27.

30. A host cell or cell free expression system comprising an expression vector according to claim 26.

31. A pharmaceutical composition comprising the isolated polypeptide according to claim 25, and pharmaceutically acceptable carriers or excipients.

32. A pharmaceutical composition comprising the nucleic acid according to claim 26 and pharmaceutically acceptable carriers or excipients.

33. A pharmaceutical composition comprising the vector according to claim 28 and pharmaceutically acceptable carriers or excipients.

34. A pharmaceutical composition comprising the vector according to claim 29 and pharmaceutically acceptable carriers or excipients.

35. A pharmaceutical composition comprising the host cell according to claim 30 and pharmaceutically acceptable carriers or excipients.

36. The pharmaceutical composition according to claim 25 for use in medicine.

37. A method of treating or preventing diabetes mellitus, insulin resistance, obesity, metabolic disorders polycystic ovary syndrome, non-alcoholic fatty liver disease, retinopathy, neuropathy, nephropathy, Alzheimer's disease or cardiovascular disease in a subject in need thereof comprising administering the composition of claim 31.


SEQUENCE LISTING (27 sequences)

SEQ ID NO: 1 - protein, 540 aa, Artificial Sequence
  VPS10p Domain- isoform 1
RGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILER

SEQ ID NO: 2 - protein, 597 aa, Artificial Sequence
  VPS10p Domain + 10CCa Domain- isoform 1
RGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSMCQNGRDYVVTKQPSICL

SEQ ID NO: 3 - protein, 623 aa, Artificial Sequence
  VPS10p Domain + 10CCb Domain- isoform 1
RGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERCSLEDFLCDFGYYRPENDSKCVEQPELKGHDLEFCLYGREEHLTTNGYRKIPGDKCQGGVNPVREVKDLKKKCTSNFLSPEKQ

SEQ ID NO: 4 - protein, 680 aa, Artificial Sequence
  VPS10p Domain + 10CCa Domain + 10CCb Domain- isoform 1
RGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSMCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQ

SEQ ID NO: 5 - protein, 57 aa, Artificial Sequence
  10CCa Domain- isoform 1
NCEEKDYTIWLAHSTDPEDYEDGCILGYKEQFLRLRKSSMCQNGRDYVVTKQPSICL

SEQ ID NO: 6 - protein, 83 aa, Artificial Sequence
  10CCb Domain- isoform 1
CSLEDFLCDFGYYRPENDSKCVEQPELKGHDLEFCLYGREEHLTTNGYRKIPGDKCQGGVNPVREVKDLKKKCTSNFLSPEKQ

SEQ ID NO: 7 - protein, 140 aa, Artificial Sequence
  10CCa + 10CCb Domain- isoform 1
NCEEKDYTIWLAHSTDPEDYEDGCILGYKEQFLRLRKSSMCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQ

SEQ ID NO: 8 - protein, 685 aa, Artificial Sequence
  IST218 after furin site plus HIS tag- isoform 1
SAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSMCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSHHHHHH

SEQ ID NO: 9 - protein, 679 aa, Artificial Sequence
  IST218 after furin site minus HIS tag- isoform 1
SAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSMCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNS

SEQ ID NO: 10 - protein, 680 aa, Artificial Sequence
  SKT1 after furin site plus HIS tag + TEV site-isoform 1
SAPGHHHHHHHENLYFQSEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSMCQNGRDYVVTKQPSICLCSLEDFLCDFGYYRPENDSKCVEQPELKGHDLEFCLYGREEHLTTNGYRKIPGDKCQGGVNPVREVKDLKKKCTSN

SEQ ID NO: 11 - protein, 662 aa, Artificial Sequence
  SKT1 after furin site minus HIS tag + TEV site- isoform 1
EDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSMCQNGRDYVVTKQPSICLCSLEDFLCDFGYYRPENDSKCVEQPELKGHDLEFCLYGREEHLTTNGYRKIPGDKCQGGVNPVREVKDLKKKCTSN

SEQ ID NO: 12 - protein, 831 aa, Homo sapiens [sequence omitted]

SEQ ID NO: 13 - protein, 830 aa, Homo sapiens
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 14 - protein, 539 aa, Artificial Sequence
  VPS10p Domain- isoform 2
RGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILER

SEQ ID NO: 15 - protein, 596 aa, Artificial Sequence
  VPS10p Domain + 10CCa Domain- isoform 2
RGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICL

SEQ ID NO: 16 - protein, 622 aa, Artificial Sequence
  VPS10p Domain + 10CCb Domain- isoform 2
RGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERCSLEDFLCDFGYYRPENDSKCVEQPELKGHDLEFCLYGREEHLTTNGYRKIPGDKCQGGVNPVREVKDLKKKCTSNFLSPEKQ

SEQ ID NO: 17 - protein, 679 aa, Artificial Sequence
  VPS10p Domain + 10CCa Domain + 10CCb Domain- isoform 2
RGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQ

SEQ ID NO: 18 - protein, 57 aa, Artificial Sequence
  10CCa Domain- isoform 2
NCEEKDYTIWLAHSTDPEDYEDGCILGYKEQFLRLRKSSVCQNGRDYVVTKQPSICL

SEQ ID NO: 19 - protein, 140 aa, Artificial Sequence
  10CCa Domain + 10CCb Domain- isoform 2
NCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQ

SEQ ID NO: 20 - protein, 684 aa, Artificial Sequence
  IST218 after furin site plus HIS tag- isoform 2
SAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSHHHHHH

SEQ ID NO: 21 - protein, 678 aa, Artificial Sequence
  IST218 after furin site minus HIS tag- isoform 2
SAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNS

SEQ ID NO: 22 - protein, 679 aa, Artificial Sequence
  SKT1 after furin site plus HIS tag + TEV site- isoform 2
SAPGHHHHHHHENLYFQSEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSN

SEQ ID NO: 23 - protein, 661 aa, Artificial Sequence
  SKT1 after furin site minus HIS tag + TEV site- isoform 2
EDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCTDLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSN

SEQ ID NO: 24 - protein, 7 aa, Artificial Sequence
  TEV cleavage site option 1
ENLYFQG

SEQ ID NO: 25 - protein, 7 aa, Artificial Sequence
  TEV cleavage site option 2
ENLYFQS

SEQ ID NO: 26 - protein, 6 aa, Artificial Sequence
  C terminal HIS tag
HHHHHH

SEQ ID NO: 27 - protein, 7 aa, Artificial Sequence
  N terminal HIS tag
HHHHHHH